(19) United States
(12) Patent Application Publication
(10) Pub. No.: US 2004/0062680 A1
(43) Pub. Date: Apr. 1, 2004

(54) Apparatus and method for generation of chlorine dioxide gas

(75) Inventor: Joel J. Kampa, Lakehills, TX (US)

Correspondence Address: SENNIGER POWERS LEAVITT AND ROEDEL, ONE METROPOLITAN SQUARE, 16TH FLOOR, ST LOUIS, MO, 63102, US

(73) Assignee: BERNARD TECHNOLOGIES, INC.

(21) Appl. No.: 10/261,037

(22) Filed: Sep. 30, 2002

(51) Int. Cl. A01N 1/00; A61L 2/00

(52) U.S. Cl. 422/29; 422/28; 422/32; 422/37; 422/40; 422/305

(57) ABSTRACT

A chlorine dioxide gas producing apparatus has a first reaction component contained therein and a second reaction component contained therein. The first and second reaction components are separated within the apparatus by at least one rupturable membrane. To activate the apparatus, the at least one rupturable membrane is ruptured to permit contact between the first and second reaction components to facilitate a chemical reaction therebetween which produces chlorine dioxide gas. The apparatus is adapted for releasing chlorine dioxide gas produced therein. The apparatus may be placed into an enclosure containing articles to be treated and the enclosure then closed to permit a concentration of chloride dioxide gas produced by the apparatus sufficient to treat the at least one article to fill the enclosure.

### BACKGROUND OF THE INVENTION

The present invention relates generally to the use of chlorine dioxide gas for various treatments such as deodorizing, sanitizing, decontaminating, sterilizing, bleaching, disinfecting and the like, and more particularly to apparatus for generating chlorine dioxide gas and to methods for using such apparatus to treat biologically contaminated surfaces and articles.

The use of gas, and more particularly chlorine dioxide gas, as a sterilizing agent, e.g., as a bactericide, viricide and sporicide, is known. For example, U.S. Pat. Nos. 4,504,442 and 4,681,739 to Rosenblatt et al. disclose the use of chlorine dioxide gas as a chemosterilizing agent. However, due the instability of chlorine dioxide as well as inherent handling difficulties associated with chlorine dioxide, apparatus used to generate chlorine dioxide gas is typically limited to fixed equipment such as a gas generator and corresponding gas chamber in which articles to be sterilized are placed. That is, reaction components which, when mixed together, produce chlorine dioxide gas must be maintained separate until gas production is desired.

As a result, articles to be sterilized must be transported to the location of the sterilizing chamber or, where a room is to be sterilized, an elaborate and costly gas producing apparatus must be transported and erected within such a room. There is a need, therefore, for apparatus for producing chlorine dioxide gas which can be readily transported to a remote site of contaminated articles, or to a contaminated room, and quickly activated to produce chlorine dioxide gas in a sufficient concentration to serve as a treating agent.

### SUMMARY OF THE INVENTION

In general, apparatus of the present invention for producing chlorine dioxide gas comprises a first reaction component and a second reaction component. The first and second reaction components are separated by at least one rupturable membrane. Upon rupturing of the at least one membrane the first and second reaction components contact each other to form a reaction in which chlorine dioxide gas is produced within the apparatus. The apparatus is also adapted for exhausting the chlorine dioxide gas therefrom.

A method of the present invention for treating at least one article contained within an enclosure generally comprises activating a chlorine dioxide producing apparatus to produce chlorine dioxide gas. The chlorine dioxide gas producing apparatus comprises a first reaction component contained therein and a second reaction component contained therein. The first and second reaction components are separated within the apparatus by at least one rupturable membrane. The step of activating the apparatus thus comprises rupturing the at least one membrane to permit contact between the first and second reaction components to facilitate a chemical reaction therebetween which produces chlorine dioxide gas within the apparatus. The apparatus is adapted for releasing chlorine dioxide gas produced therein. The apparatus is placed into the enclosure and the enclosure is closed to permit a concentration of chloride dioxide gas produced by the apparatus sufficient to treat the at least one article to fill the enclosure.

In another embodiment a method for treating postal articles generally comprises placing at least one postal article in a bag and activating a chlorine dioxide producing apparatus to generate chlorine dioxide gas. The chlorine dioxide producing apparatus is placed in the bag and the bag is closed such that a concentration of chlorine dioxide gas sufficient to treat the at least one postal article fills the bag.

### BRIEF DESCRIPTION OF THE DRAWINGS

FIG. 1 is a cross-section of a first embodiment of apparatus of the present invention for producing chlorine dioxide gas;

FIG. 2 is a cross-section of a second embodiment of apparatus of the present invention;

FIG. 3 is a side elevation of a third embodiment of apparatus of the present invention with a pouch of the apparatus shown open and with portions cut away to reveal internal construction;

FIG. 4 is a cross-section of a fourth embodiment of apparatus of the present invention;

FIG. 5 is a cross-section of a fifth embodiment of apparatus of the present invention;

FIG. 6 is a cross-section of a sixth embodiment of apparatus of the present invention;

FIG. 7 is a cross-section of a seventh embodiment of apparatus of the present invention;

FIG. 8 is a graph of chlorine dioxide gas concentration versus time for one apparatus of the present invention;

FIG. 9 is a graph of chlorine dioxide gas concentration versus time for one apparatus of the present invention tested with various amounts of reaction components;

FIG. 10 is a graph of chlorine dioxide gas concentration versus time for various apparatus of the present invention;

FIG. 11 is a graph similar to that of FIG. 10 for an extended duration; and

FIG. 12 is a graph of chlorine dioxide gas concentration versus time for one apparatus of the present invention tested with various reaction components.

Corresponding reference characters indicate corresponding parts throughout the several views of the drawings.

### DETAILED DESCRIPTION OF THE INVENTION

The apparatus of the present invention for producing and releasing chlorine dioxide gas (e.g., ClO₂) for use as a treating agent, such as for deodorizing, sanitizing, decontaminating, sterilizing, bleaching, disinfecting and the like, relies on the separate containment of two or more reactive components during transport to a remote location, followed by activation of the apparatus to permit chemically reactive mixing of the components to form a reaction in which a chlorine dioxide gas is produced and released from apparatus. The reactive components may be any combination of reactants capable of reacting to form chlorine dioxide gas.

Chlorine dioxide gas may be produced by mixing a first reaction component such as an acid releasing agent , an oxidizing agent or a mixture thereof with a second reaction component comprising a source of chlorite anions to form chlorine dioxide by acidification and/or oxidation of the chlorite source. For example, chlorine dioxide gas may be produced by the acidification of sodium chlorite (e.g., NaClO₂) according to the following reaction:

4 H⁺+5 NaClO₂→4 ClO₂+2 H₂O+5Na⁺+Cl⁻ Eq. 1

or by the oxidation of sodium chlorite by persulfate, according to the following reaction:

2 NaClO₂+NaS₂O₈→2 ClO₂+2 Na₂SO₄ Eq. 2

Suitable chlorite sources include, for example, alkali metal chlorites such as sodium chlorite or potassium chlorite, alkaline-earth metal chlorites such as calcium chlorite, or chlorite salts of a transition metal ion or a protonated primary, secondary, tertiary or quaternary amine such as ammonium chlorite, trialkylammonium chlorite and quarternary ammonium chlorite.

The acid releasing agent may be any acid or substance that can be hydrolyzed to an acid which is capable of reacting with the chlorite source to form chlorine dioxide. Suitable acid releasing agents include, for example, carboxylic acids, anyhydrides, acyl halides, phosphoric acid, phosphate esters, trialkylsilyl phosphate esters, dialkyl phosphates, poly phosphates, condensed phosphates, sulfonic acid, sulfonic acid esters, sulfonic acid chlorides, phosphosilicates, phosphosilicic anhydrides, carboxylates of poly α-hydroxy alcohols such as sorbitan monostearate or sorbitol monostearate, phophosiloxanes, hydrochloric acid, boric acid, citric acid, malic acid, tartaric acid, mineral acids and metal salts with sufficiently acid aqueous ions such as zinc, aluminum and iron. It is understood that other acid sources may be used, but is preferably selected to cause the mixture of reactants to have a pH equal to or less than about 5.5.

Suitable oxidizing agents are any oxidizing agent which is a stronger oxidation potential than the chlorite source such as, for example, persulfate, chlorine gas and the like.

The reaction components of the apparatus of the present invention may each be in the form of a gas, a liquid, or a solid, or a combination of gas, liquid and/or solid. For example, in one reaction according to Eq. 1, one reaction component is a liquid solution prepared from sodium chlorite solution and sodium silicate solution and the other reaction component is an acid, such as hydrochloric acid, in either a liquid or solid form. In another embodiment, such as in accordance with Eq. 2, one reaction component is a liquid solution of sodium chlorite and the other reaction component is a mixture of sodium persulfate (e.g., Na₂S₂O₈) powder in a silica gel.

As will be described in further detail below, the reaction components are generally contained in separate chambers within the apparatus with a rupturable membrane therebetween for safe and convenient transport of the reaction components to a remote site. The chlorine dioxide gas is produced by rupturing the membrane to permit reactive mixing of the reaction components within the apparatus and is then released from the apparatus. The rate at which the chlorine dioxide gas is released from the apparatus is generally a function of the rate at which the reaction components mix within the apparatus, the rate at which the reaction to produce the chlorine dioxide gas occurs and the rate at which the particular construction of the apparatus permits the chlorine dioxide gas to be released therefrom. The concentration and amount of chlorine dioxide gas to be produced is generally a function of the concentration and quantity of the reaction components, the completeness of the reaction and the size of an enclosed area to be treated.

The rate at which the chlorine dioxide is produced and exhausted from the apparatus may be further affected by adding one or more adjuvant(s) to the first reaction component and/or the second reaction component. More precisely, by adding the appropriate adjuvant to the first and/or second reaction component(s), the rate at which the reactants are available to the reaction may be reduced thereby reducing the rate at which the chlorine dioxide gas is produced. This may also reduce the rate at which the chlorine dioxide gas is exhausted from the apparatus and inhibit liquid in the apparatus following mixing of the reaction components against spilling or otherwise leaking out of the apparatus. For example, one or more absorbent(s) may be added to either or both of the reaction components. The absorbent may reduce the rate at which the reaction occurs by simply diluting the concentration of the reactants and/or by absorbing one or more of the reactants thereby suppressing the rate at which the reactants contact each other by requiring one or both of the reactants to desorb from the absorbent prior to contacting the other reactant. In addition, an absorbent added to either reaction component may affect the rate at which the chlorine dioxide gas is evolved by causing the chlorine dioxide gas produced by the reaction to be partially or completely absorbed into the absorbent and then desorbed over time. Typical absorbents include zeolites, woven and non-woven and non-powdered polymers, natural fibers (e.g., cotton, sawdust or other cellulosic materials), and inorganic materials such glass wool and clays (including hydrophobic and hydrophillic clays.

Other diluents which do not absorb either the reaction components or chlorine dioxide gas product may be added to dilute the concentration of the reactants and therefore reduce the rate at which the reaction occurs. Typical diluents include water, silica gel, clays (including hydrophobic and hydrophillic clays), zeolites, metal oxides, carbides, nitrides and glass fibers.

Finally, the rate at which the chlorine dioxide gas is evolved may be increased by adding additional reactants to the first and/or second reaction component to cause the co-generation of one or more gaseous product(s) such as, for example, carbon dioxide or nitrogen which act as a propellant increasing the rate at which the chlorine dioxide gas evolves from the apparatus.

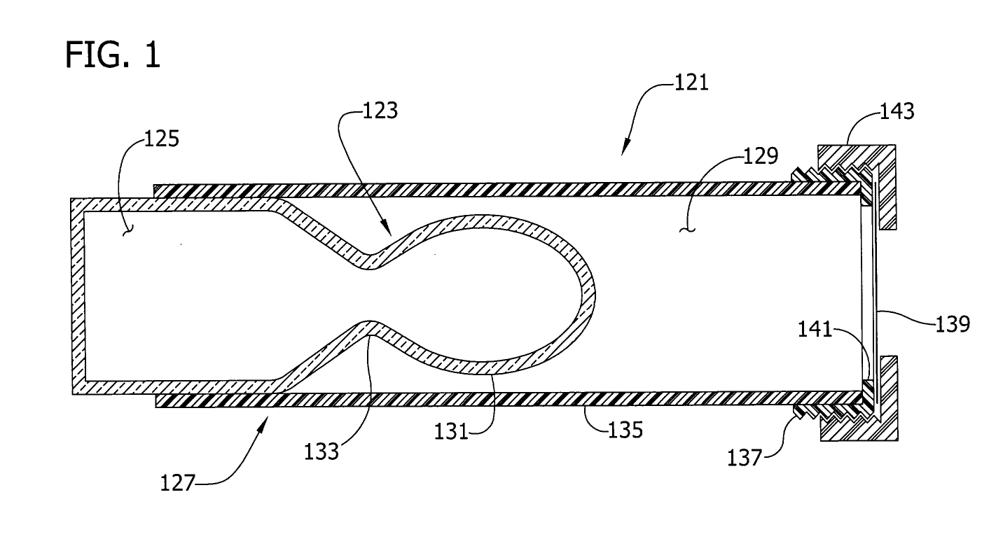

With reference now to the drawings, and in particular to FIG. 1, apparatus of the present invention for producing and releasing chlorine dioxide gas is indicated in its entirety by the reference numeral 121. The apparatus 121 comprises a first container, generally indicated at 123, defining a first chamber 125 for containing the first reaction component, and a second container, generally indicated at 127, surrounding the first container and defining a second chamber 129 for containing the first container and the second reaction component. The wall of the first container 123 is desirably rupturable, such as by being constructed of thin glass, to broadly define a rupturable membrane separating the first and second chambers 125, 129 whereby rupture of the membrane permits chemically reactive contact between the reaction components to produce chlorine dioxide gas within the second chamber. As an example, the first container 123 of the illustrated embodiment comprises a small ampule 131 constructed of thin glass and having a narrowed neck 133. The ampule 131 may be scored at its neck 133 so that the neck is easily broken upon application of a bending force thereto. It is contemplated that the ampule 131 may also be constructed of a material other than glass, such as a polymeric material, as long as the material is easily ruptured and is substantially chemically non-reactive with the reaction components of the apparatus 121.

The second container 127 of the illustrated embodiment comprises a tube 135 having an inner diameter sized for receiving the ampule 131 therein, neck 133 end first, in generally sealing engagement with the tube to seal one end of the tube. The tube 135 is desirably flexible to permit bending thereof and is constructed of a generally gas and liquid impermeable material. For example, one preferred such material is polyvinyl chloride (PVC). An annular end cap 137 is fitted on the opposite end of the tube 135 and a closure 139 constructed of a gas permeable but liquid impermeable material is secured over a central opening 141 of the end cap. More particularly, the end cap 135 of the illustrated embodiment is constructed of glass and has exterior threads formed therein. The closure 139 is constructed of a single layer of a material available from Du Pont de Nemours of Wilmington, Delaware under the tradename Tyvek® and is secured to the end cap 135 over the central opening 141 by an annular retaining ring 143 adapted for threaded engagement with the exterior threads of the end cap.

To construct the apparatus 121 of FIG. 1, the ampule 131 is filled with a first reaction component, such as a sodium chlorite solution, and sealed. For example, the ampule may be filled in the range of about 66 percent to about 75 percent of its volumetric capacity and then flame sealed. The ampule 131 is then fitted snugly into one end of the tube 135 to seal that end of the tube. A second reaction component, such as a mixture of sodium persulfate powder (Na₂S₂O₈) and silica gel, is loaded through the other end of the tube 135 into the interior thereof. The end cap 137 is then fitted onto the open end of the tube 135 and the closure 139 is secured over the central opening 141 of the end cap by the retaining ring 143.

In operation according to one method of the present invention for producing and releasing chlorine dioxide gas, the apparatus 121 is activated by flexing the tube 135 to apply a bending force to the ampule 131, thereby breaking the ampule at its neck 133. More broadly stated, the rupturable membrane (e.g., the wall of the first container 125) separating the first and second reaction chambers 125,129 within the apparatus is ruptured. The operator then shakes the apparatus 121 to cause the reaction component in the ampule 131 to flow into the interior of the tube 135 for chemically reactive contact with the silica mixture. The solution is absorbed by the silica mixture, resulting in a semi-solid mixture which produces chlorine dioxide gas within the tube 135. Chlorine dioxide gas is exhausted from the apparatus 121 through the gas permeable closure 139. While the rate at which gas is exhausted from the apparatus 121 may be controlled by the gas permeability of the closure 139, the gas permeability of the closure 139 is desirably sufficient to allow gas to permeate therethrough at a rate substantially equal to or greater than the rate at which chlorine dioxide gas is produced within the tube 135. It is understood, however, that the gas permeability of the closure 139 may inhibit the exhaustion of gas from the tube 135 at the same or higher rate at which the gas is produced, as long as the tube, end cap 137, closure 139 and retaining ring 143 are sufficiently constructed and arranged to withstand the corresponding gas pressure build-up within the tube.

It is contemplated that the ampule 131 containing the first reaction component may be ruptured by mechanical stimuli other than bending, such as by applying compression (e.g., by squeezing the tube 135 and the ampule therein), pushing, pulling and/or shaking, by an ultrasonic stimuli, by an electromagnetic stimuli (e.g., electrical, infrared and the like), a thermal stimuli or other suitable stimuli for rupturing the ampule without departing from the scope of this invention.

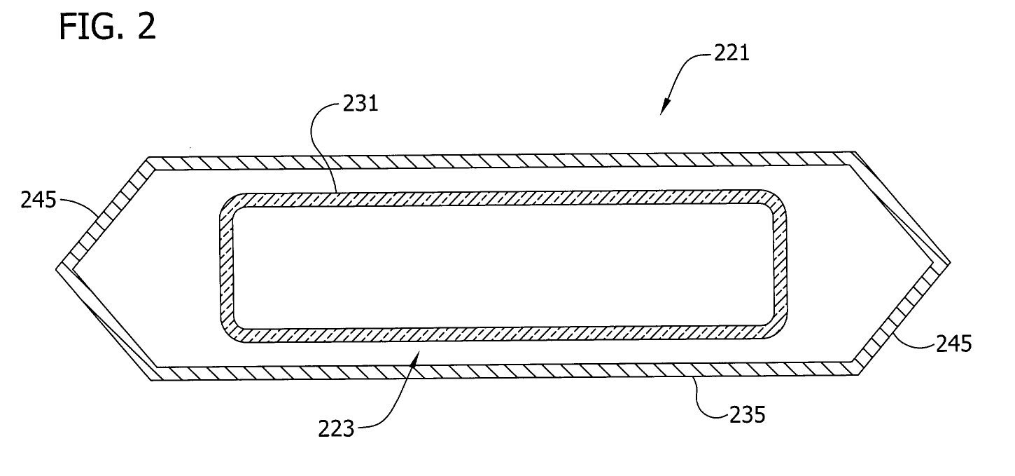

FIG. 2 illustrates a second embodiment of apparatus 221 of the present invention in which the first container 223 comprises a generally tubular ampule 231 having sealed ends. The ampule 231 is constructed of a thin-walled glass, also sometimes referred to as “onion skin” glass, so that it can be easily ruptured upon application of a compression (e.g., squeezing) force or a bending force thereto. For example, one such thin-walled glass is available from Kimble of Chicago, Ill. The second container 227 comprises a flexible tube 235 constructed of a generally gas permeable but liquid impermeable material. For example, one preferred such material from which the tube 135 may be constructed is available from Du Pont de Nemours under the tradename Teflon®. The wall thickness of the tube 235 is desirably sufficient to provide a slow or otherwise controlled diffusion of gas therethrough while sufficiently withstanding bending of the tube as well as gas pressure build-up within the tube. As an example, the wall thickness of the tube 235 may be approximately 0.125 inches.

To construct the apparatus 221 of this second embodiment, the ampule 231 is filled with a first reaction component, such as concentrated hydrochloric acid (liquid), and sealed. One end of the flexible tube 235 is closed, such as by being heat sealed, and the filled ampule 231 is inserted through the other, open end of the tube into the interior of the tube. A second reaction component, such as a solution prepared from equal parts of a sodium chlorite solution and a sodium silicate solution, is dispensed into the interior of the tube 235 and the open end of the tube is then closed, such as by being heat sealed, to fully enclose the filled ampule 231 and the second reaction component within the tube.

It is contemplated that the ampule 231 may be of any shape, such as ovate, spherical, etc., and may have narrowed and/or scored portions similar to the neck of the ampule shown in FIG. 1, without departing from the scope of this invention. The relative sizes of the tube 235 and ampule 231 is generally dependent on the desired volumes of the first and second reaction components. In one embodiment, the tube 235 and ampule 231 are both tubular wherein the tube has an aspect ratio (e.g. tube length to tube inner diameter) of less than or equal to about 12 to facilitate efficient mixing of the reaction components and the ampule takes up no more than about one-half of the volumetric capacity of the tube. For example, the tube may have a length of about six inches and an inner diameter of about 0.5 inches.

In operation, the apparatus 221 is activated by bending the flexible tube 235 to apply a bending force to the ampule 231 to thereby rupture the ampule. More preferably, the tube 235 is bent repeatedly to cause several breaks along the length of the ampule 231. The apparatus 221 is then shaken vigorously to cause the first reaction component contained in the ampule 231 to mix with the second reaction component within the tube 235. The mixing results in a rapid precipitation of the silicate, leaving a generally solid mixture within the tube 235 whereby chlorine dioxide gas is produced as the mixture becomes acidic. The chlorine dioxide gas is exhausted from the apparatus 221 by diffusing out through the gas permeable wall of the tube.

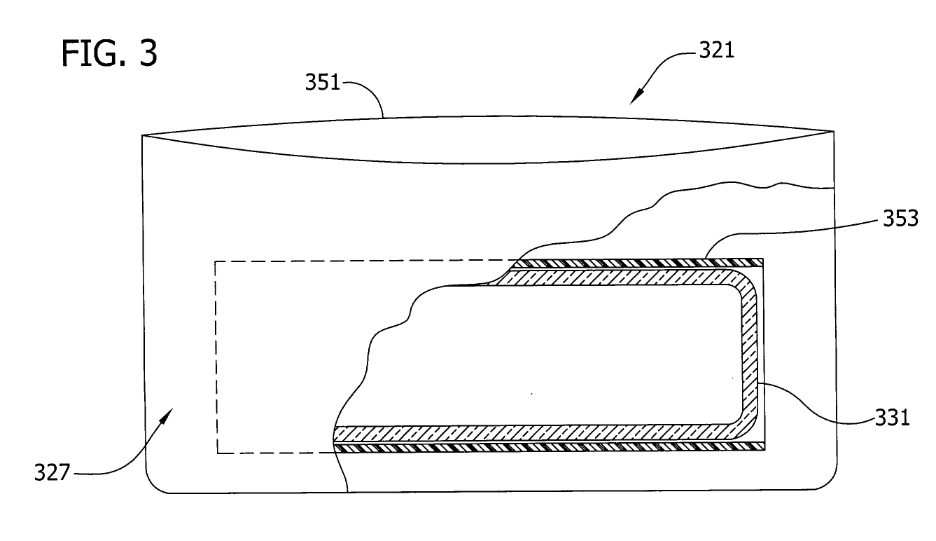

In a third apparatus 321 of the present invention as shown in FIG. 3, a glass ampule 331 similar to that of the second embodiment of FIG. 2 is placed in a second container 327 comprising a pouch 351. The pouch 351 is preferably constructed of a flexible, gas permeable but liquid impermeable material to permit chlorine dioxide gas generated within the pouch to permeate outward therefrom for exhaustion from the apparatus 321. For example, the pouch 351 of the illustrated embodiment is constructed of a pair of sheets constructed of a flexible, gas permeable material and heat sealed together along three sides (e.g. the bottom and sides of the illustrated embodiment) thereof to define the interior of the pouch. More desirably, the material from which the pouch 351 is constructed is desirably sufficient to allow gas to permeate therethrough at a rate substantially equal to or greater than the rate at which chlorine dioxide gas is produced within the pouch. It is understood, however, that the gas permeability of the material may inhibit the exhaustion of gas from the pouch 351 at the same or higher rate at which the gas is produced, as long as the pouch is sufficiently constructed to withstand the corresponding gas pressure build-up therein. One preferred material from which the pouch may be constructed is available from Du Pont De Nemours of Wilmington, Del. under the tradename Tyvek® and has a thickness of about 5 mil.

A protective liner 353 surrounds the glass ampule 331 within the pouch 351 to protect the pouch against puncture by glass shards while rupturing the ampule. One preferred such protective liner 353 is constructed of a sheet of PVC having a thickness of about 5 mil and is formed, e.g., rolled, into a generally tubular configuration. The protective liner 353 may alternatively be constructed of a polyethylene or other polymer sheet, a woven mesh or other suitable material as long as it is sufficiently flexible to allow breaking of the ampule 331 within the pouch 351.

The apparatus 321 is assembled by first forming the pouch as described above. The ampule 331 is filled with a first reaction component, such as a sodium chlorite solution, and sealed. The protective liner 353 is formed into a generally tubular configuration around the ampule 331 and the liner and ampule are together placed inside the pouch 351 along with a mixture of sodium persulfate powder and silica gel as described above with respect to the first embodiment of FIG. 1. The open side of the pouch is then closed, such as by being heat sealed.

The apparatus 321 is activated by crushing the ampule 331, such as by squeezing or bending the pouch 351, to permit the sodium chlorite solution to leak from the ampule into the interior of the pouch. The sodium chlorite solution contacts and reacts with the mixture contained in the pouch 351 to produce chlorine dioxide gas therein. The chlorine dioxide gas diffuses out from the apparatus 321 through the gas permeable walls of the pouch 351 while remaining liquid is absorbed by the silica and is inhibited against leaking out of the pouch, e.g., since the walls of the pouch are liquid impermeable.

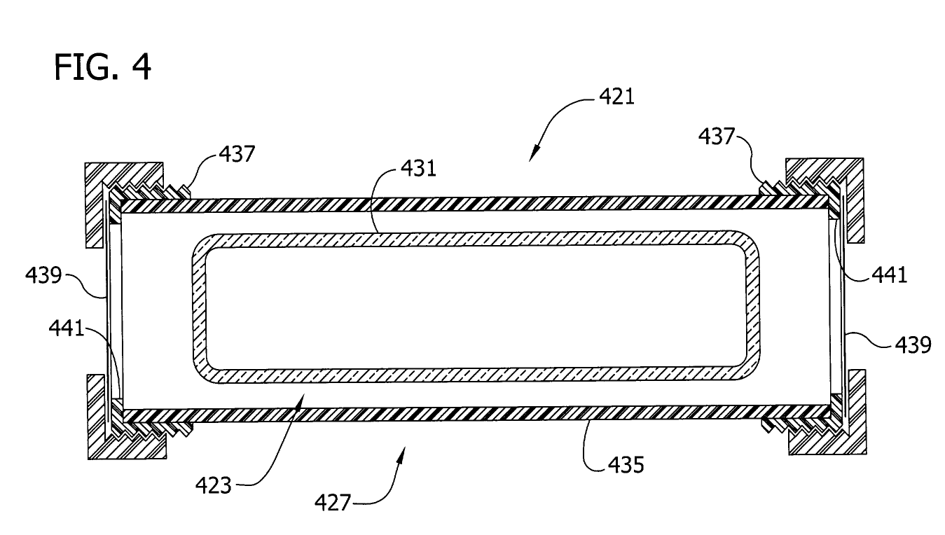

With reference now to FIG. 4, the first container 423 of a fourth embodiment of apparatus 421 of the present invention is a glass ampule 431 substantially similar to that of the second embodiment of FIG. 2. The second container 427 comprises a tube 435 constructed of a flexible, gas and liquid impermeable material. For example, the tube 435 of the illustrated embodiment is constructed of PVC (e.g., Tygon®) having a length and an inner diameter sized for fully receiving the ampule therein. For example, the relative sizes of the ampule and tube may be substantially the same as described previously for the apparatus 221 of the second embodiment. End caps 437 similar to the end cap 137 of the first embodiment (FIG. 1) are secured to each end of the tube 435 and closures 439 constructed of one or more layers of gas permeable but liquid impermeable material are secured over the central openings 441 of the end caps. As an example, one preferred such material from which the closures may be constructed is Tyvek®. It is understood that only one end cap 437 may be provided, with the other end of the tube 435 being sealed, without departing from the scope of this invention.

To construct the apparatus of this fourth embodiment, the ampule 431 is filled with a first reaction component, such as a sodium chlorite solution, and sealed. One end cap 437 is secured to an end of the tube 435 in sealing engagement therewith and a closure 439 is secured over the central opening 441 of the end cap. The ampule 431 is then inserted through the open end of the tube 435 into the interior thereof and a second reaction component, such as a mixture of sodium persulfate powder and silica gel is dispensed into the tube. The other end cap 437 and closure 439 are then secured to the open end of the tube 435 in sealing engagement therewith to seal the ampule 431 and second reaction component within the interior of the tube. The apparatus 421 is activated by repeatedly bending the tube 435 to break the ampule 431, thereby permitting chemically reactive contact between the reaction components. Chlorine dioxide gas is thus produced and exhausted from the apparatus 421 by diffusing through the gas permeable closures 439 at the ends of the tube.

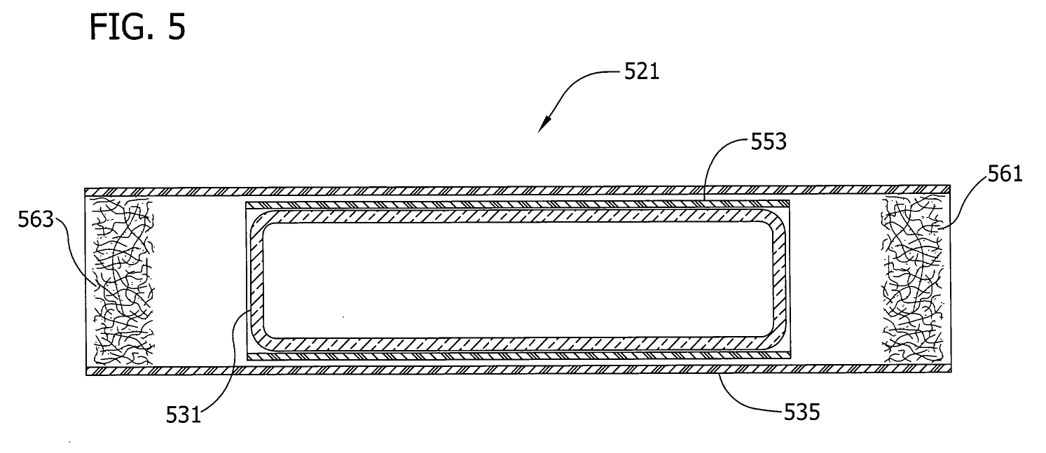

A fifth embodiment of apparatus 521 of the present invention as shown in FIG. 5 is similar in construction to that of the fourth embodiment (FIG. 4), but with the tube 535 instead being constructed of a heat shrink material adapted for shrinking upon application of heat thereto. For example, one material from which the tube 535 may be constructed is polyethylene. After the ampule 531 is filled and sealed, the ampule is placed within a generally tubular protective sheath 553 to protect the tube 535 against damage from glass shards upon rupturing of the ampule. As an example, the protective sheath 553 is desirably constructed of woven nylon but may be constructed of the same materials as the liner 353 of the third embodiment (FIG. 3) or other suitable materials as long as the sheath is sufficiently flexible to permit rupturing of the ampule 531 upon flexing the tube 535. A plug 561 constructed of glass wool is stuffed into one end of the tube 535 and the ampule 531, sheath 553 and mixture of sodium persulfate powder and silica gel are inserted through the other end of the tube into the interior thereof. Another glass wool plug 563 is stuffed into the other end of the tube 535 and the entire apparatus 521 is heated, such as by using a heat gun, to shrink the tube around the ampule 531 and glass wool plugs 561, 563. The apparatus is heated until the glass wool plugs 561, 563 are firmly held in place within the tube 535. In one embodiment, the tube 535 has an inner diameter of about 0.375 inches prior to heating and shrinks to about 0.25 inches following heating of the tube. Chlorine dioxide gas generated upon activation of the apparatus 521 is exhausted through the glass wool plugs 561, 563 at the ends of the tube 535.

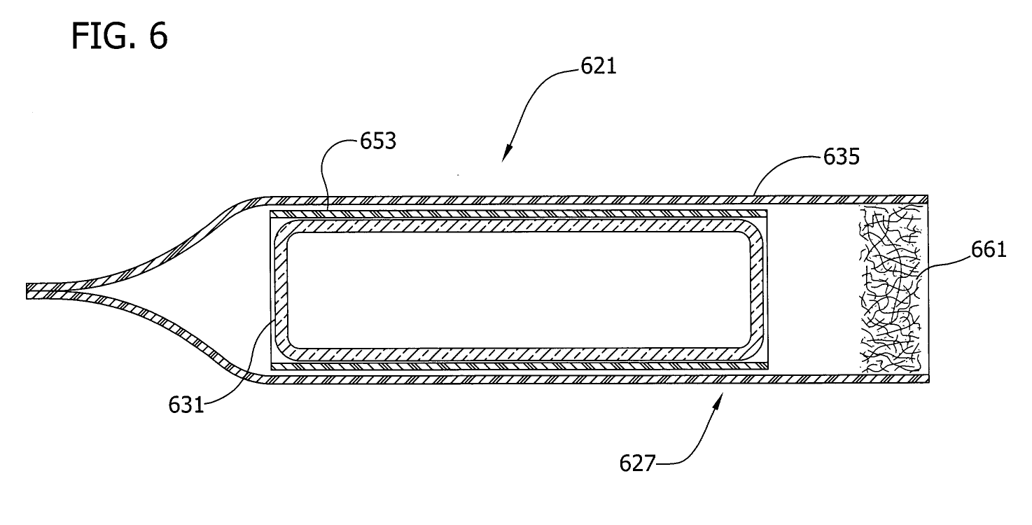

In a sixth embodiment of apparatus 621 (FIG. 6) of the present invention, the second container 627 comprises a tube 635 configured to have an appearance similar to that of a toothpaste tube. The tube 635 is preferably constructed of a flexible, gas permeable but liquid impermeable material. For example, one such material from which the tube 635 may be constructed is PVC or Tyvek®. The tube 635 is initially formed such that the diameter of the tube increases slightly from one end to the other. A glass wool plug 661 is inserted into the larger diameter end of the tube 635 and pushed therethrough to wedge the plug within the tube adjacent the smaller diameter end. A filled and sealed ampule 631 is surrounded by a generally tubular protective sheath 653, such as the sheath 553 of FIG. 5, and the ampule and sheath are together inserted through the large diameter end of the tube 635 into the interior thereof. The second reaction component, such as a sodium persulfate and silica gel mixture, are added to the interior of the tube 635 and the open end of the tube is then closed, such as by being heat-sealed. Activation and operation of the apparatus 621 is substantially the same as the apparatus 521 of the fifth embodiment (FIG. 5) described above.

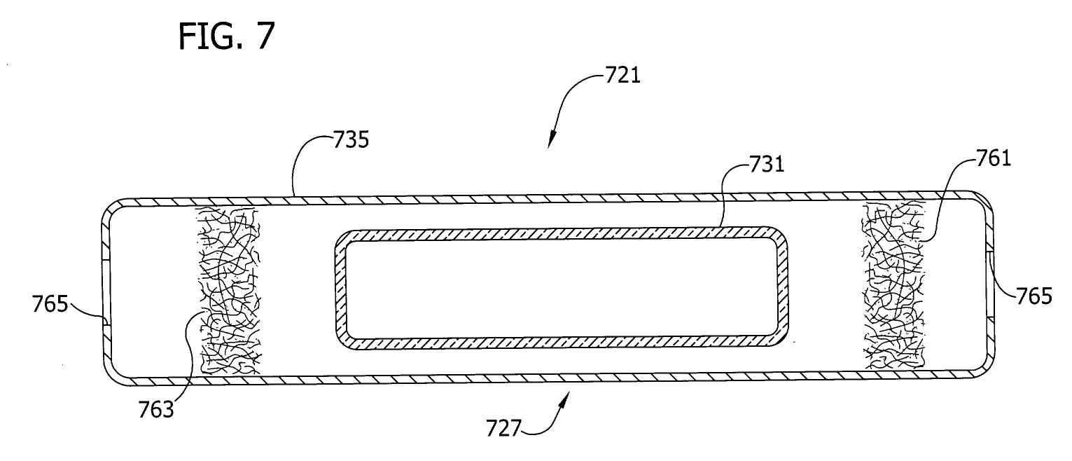

FIG. 7 illustrates a seventh embodiment of apparatus 721 of the present invention in which the second container 727 comprises a tube 735 constructed of a flexible, gas permeable but liquid impermeable material. As an example, one preferred such material is Teflon®. The tube 735 is closed at one end, such as by being heat sealed, to form a generally rounded end. A glass wool plug 761 is inserted into the tube 735 via the open end thereof and pushed through the tube to adjacent its sealed end. A filled and sealed ampule 731 is inserted into the tube 735 along with a second reaction component, such as a sodium persulfate and silica gel mixture. A second glass wool plug 763 is then inserted into the open end of the tube 735 and the open end is closed, such as by being heat sealed. Small holes 765 are formed in each end of the tube, such as by being drilled therein. Upon activation of the apparatus 721, chlorine dioxide gas is exhausted from the tube by passing out through the glass wool plugs 761, 763 and holes 765 as well as by diffusing out through the gas permeable wall of the tube 735.

Experiment 1

Apparatus 121 of the first embodiment described above and shown in FIG. 1 were constructed with each glass ampule 131 filled with about 5 grams of a 20% sodium chlorite solution. Along with the ampule 131, the interior of the tube 135 was filled with 5.3 grams of a mixture of 25% sodium persulfate (powdered) in silica gel (e.g., 200-400 mesh, 60 Å). The tube 135 of each apparatus 121 was constructed of polyvinyl chloride (PVC) and the closure 139 covering the central opening 141 of the end cap 137 was constructed of a single layer of Tyvek®.

The effectiveness of the apparatus 121 in a generally cold sterilization application was evaluated using biological indicators to confirm sterilization. More particularly, each apparatus 121 was placed in a sterilization bag along with two humidification sources (e.g., such as are commonly available from H. W. Andersen Products, Inc. of North Carolina, U.S.A. under the trade name Humidichips), a biological indicator, and two minor packs, each having gas permeable outer walls and containing three biological indicators as well as various medical devices and materials to be sterilized. The sterilization bag was placed in a sterilization chamber and pre-conditioned for four hours at about 50° C. The apparatus 121 was then activated within the sterilization bag to generate and disperse chlorine dioxide gas within the bag. Sterilization continued for about 15.25 hours. After consecutive purge cycles of about 0.5 hours and 0.25 hours, respectively, the biological indicators were removed and incubated for about 48 hours. Inspection of the biological indicators removed from the sterilization bags indicated sterility (e.g., >6 logs kill) in all of the biological indicators.

Experiment 2

Apparatus 221 of the type described above in connection with the second embodiment and shown in FIG. 2 were constructed in two different sizes. In the smaller sized apparatus 221, the glass ampule 231 contained about 0.4 ml of a solution prepared from equal amounts of 30% sodium chlorite solution and 2.5 ratio sodium silicate solution (e.g., 14% NaOH). The ampule 231 was placed in the tube 235 along with about 0.7 grams of 33% (in H₂O) sodium persulfate. The larger sized apparatus 221 comprised a glass ampule 231 containing about 2 ml of the sodium chlorite and sodium silicate solution and the tube 235 contained about 4 grams of the sodium persulfate.

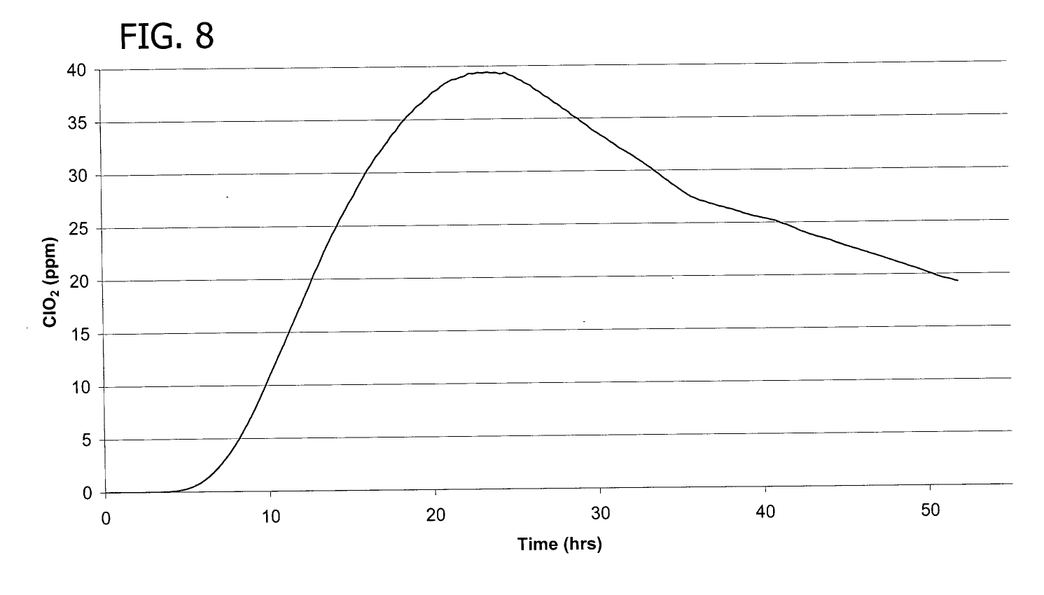

The apparatus 221 were activated and placed in separate 16 oz. jars each having a lid fitted with an electrochemical sensor capable of monitoring the chlorine dioxide concentration within the jar. FIG. 8 is a graph of the chlorine dioxide concentration (parts per million) versus time (hours) for the smaller sized apparatus 221. The smaller apparatus 221 resulted in a delay of about five hours before chlorine dioxide concentration began to build within the test jar. Thus, the relatively thick walls of the apparatus 221 result in a considerable barrier to the diffusion of chlorine dioxide gas from the apparatus, thereby providing a more controlled release of the gas over several days.

Experiment 3

Apparatus 321 of the type described above with respect to the third embodiment and shown in FIG. 3 were constructed to have different concentrations and amounts of the reaction components in accordance with the following table.

| | NaClO₂ | NaClO₂ | | Na₂S₂O₈ |
|---|---|---|---|---|
| Sample | Concentration | Solution | Na₂S₂O₈ | Mix |
| ID | (%) | Mass (g) | Concentration (%) | Mass (g) |
| 1 | 20 | 0.5 | 25 | 0.7 |
| 2 | 20 | 1 | 25 | 1.2 |
| 3 | 30 | 2 | 50 | 1.6 |

For each apparatus 321, the glass ampule 331 was filled with the specified amount and concentration of sodium chlorite solution and placed in a tubular protective liner 353 constructed from a PVC sheet having a thickness of about 5 mil. The liner 353 and ampule 331 were together placed in a pouch 351 constructed from Tyvek®, as described previously, along with the specified amount and concentration of sodium persulfate and silica gel mixture. Each apparatus 321 was tested by activating the apparatus and placing it in a sealable polyethylene (e.g., gas impermeable) bag, having a size of about 28 inches by 32 inches, along with several postal articles including a box, a 9 inch×12 inch envelope and a standard 4 inch×9 inch envelope.

The bag and postal articles were configured to allow sampling of the chlorine dioxide gas within the bag and within each article therein by a gas-tight syringe inserted through a septum port of the bag. The chlorine dioxide gas was sampled via the syringe and immediately injected into a vial containing 20 ml of solution prepared from 1% potassium iodide (KI) solution and 5 ml of acetic acid. The resulting iodine was titrated using sodium thiosulfate and a starch indicator.

The table below identifies the chlorine dioxide concentration, in parts per million (ppm) measured within the bag enclosure for each of the three variations of apparatus 321 tested.

| | Measured ClO₂ Concentration |
|---|---|
| Sample ID | (ppm) |
| 1 | 180 |
| 2 | 448 |
| 3 | 1344 |

Experiment 4

As a further test, additional apparatus 321 of the type described above with respect to the third embodiment and as shown in FIG. 3 were constructed in accordance with the reaction component concentrations and amounts identified in the following table.

| | | NaClO₂ | Na₂S₂O₈ | Na₂S₂O₈ |
|---|---|---|---|---|
| Sample | NaClO₂ | Solution | Concentration | Mix |
| ID | Concentration. (%) | Mass (g) | (%) | Mass (g) |
| 1 | 30 | 0.237 | 50 | 0.180 |
| 2 | 30 | 0.508 | 50 | 0.385 |
| 3 | 30 | 0.523 | 50 | 0.397 |
| 4 | 30 | 0.556 | 50 | 0.422 |
| 5 | 30 | 0.915 | 50 | 0.694 |
| 6 | 30 | 1.023 | 50 | 0.776 |
| 7 | 30 | 1.047 | 50 | 0.794 |
| 8 | 30 | 1.195 | 50 | 0.906 |
| 9 | 30 | 1.506 | 50 | 1.228 |
| 10 | 30 | 1.62 | 50 | 1.142 |
| 11 | 30 | 1.692 | 50 | 1.283 |
| 12 | 30 | 2.484 | 50 | 1.883 |
| 13 | 30 | 2.81 | 50 | 2.131 |
| 14 | 30 | 2.878 | 50 | 2.182 |
| 15 | 30 | 4.082 | 50 | 3.095 |

For each apparatus 321, the glass ampule 331 was filled with a sodium chlorite solution in the specified concentration and amount and was inserted into a tubular protective liner 353 constructed from a PVC sheet having a thickness of about 5 mil. The liner 353 and ampule 331 were together placed in a pouch 351 constructed of Tyvek®, as described previously, along with the sodium persulfate and silica gel mixture in the specified concentration and amount.

Each apparatus 321 was activated and placed in a 12.8 liter glass flask and the flask was sealed with a tight fitting rubber stopper. A gas tight syringe was inserted through a septum covered syringe port of the stopper to periodically remove a sample of chlorine dioxide gas from the flask. The resulting chlorine dioxide concentration within the flask was then determined by iodometric titration as described previously in Experiment 3. The concentration in each flask was sampled for a period of about 1.5 hours. However, for one tested apparatus 321 the concentration was sampled over a period of about four hours to illustrate the persistence of the chlorine dioxide gas concentration in the flask, without further generation of the gas.

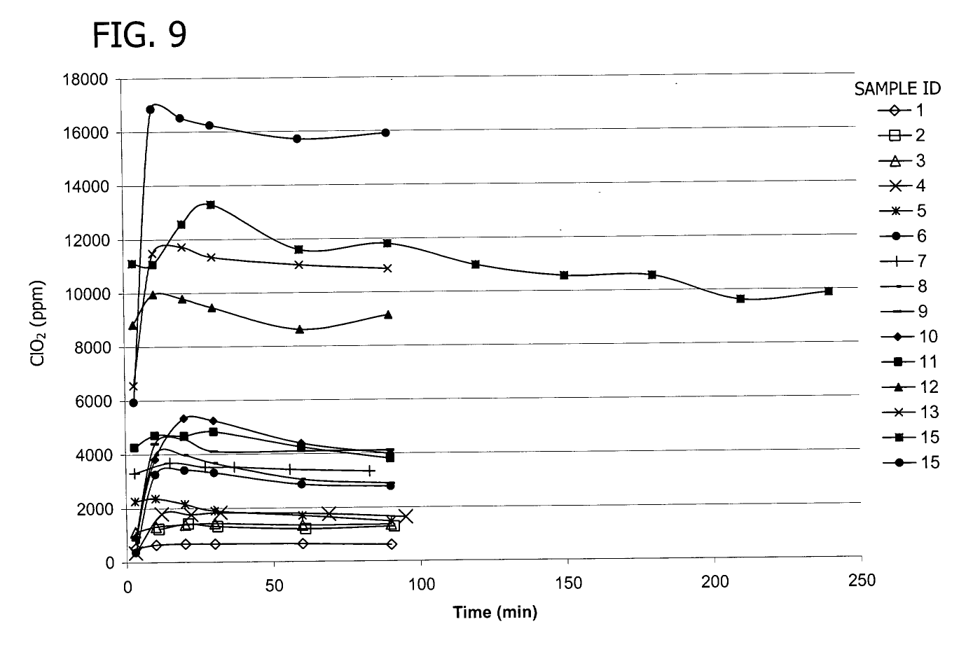

FIG. 9 is a graph of chlorine dioxide concentration (parts per million) within the flask versus time (minutes). As is evident from the graph, the concentration of chlorine dioxide gas within the flask increased with the mass of sodium chlorite and sodium persulfate present in the apparatus 321.

Experiment 5

Another experiment was conducted to determine the effect of various apparatus constructions of the present invention on the production of chlorine dioxide gas. The experiment also evaluated the effect on chlorine dioxide gas production of using different combinations of reaction components and reaction component concentrations in the apparatus of the present invention. To conduct the experiment, various apparatus 321, 421, 521, 621, 721 of the types described above and shown in FIGS. 3, 4, 5, 6 and 7 were constructed in accordance with the following table.

| | | NaClO₂ | | | | |
|---|---|---|---|---|---|---|
| | | Soln. | | Co-Reactant Mixture | | |
| | Apparatus | Conc. | Vol. | | Conc. | Mass |
| Sample ID | Type | (%) | (ml) | Acid/Oxidant | (%) | (g) |
| 1 | 321 (FIG. 3) | 30 | 1 | Na₂S₂O₈ | 50 | 1 |
| 2 | 321 (FIG. 3) | 30 | 1 | Na₂S₂O₈ | 50 | 1 |
| 3 | 421 (FIG. 4) | 30 | 1 | Na₂S₂O₈ | 50 | 1 |
| 4 | 521 (FIG. 5) | 30 | 1 | Na₂S₂O₈ | 50 | 1 |
| 5 | 621 (FIG. 6) | 30 | 1 | Na₂S₂O₈ | 50 | 1 |
| 6 | 721 (FIG. 7) | 30 | 2 | Na₂S₂O₈ | 25 | 4 |
| 7 | 721 (FIG. 7) | 30 | 0.4 | Na₂S₂O₈ | 50 | 1 |
| 8 | 321 (FIG. 3) | 5 | 1 | Na₂S₂O₈ | 25 | 0.4 |
| 9 | 321 (FIG. 3) | 30 | 1 | Boric Acid | 50 | 1 |
| 10 | 321 (FIG. 3) | 30 | 1 | NaH2PO4 | 50 | 1 |
| 11 | 321 (FIG. 3) | 30 | 1 | Citric Acid | 50 | 1 |
| 12 | 321 (FIG. 3) | 30 | 1 | Malic Acid | 50 | 1 |
| 13 | 321 (FIG. 3) | 30 | 1 | Tartaric Acid | 50 | 1 |
| 14 | 321 (FIG. 3) | 30 | 1 | Poultry Guard | Neat | 2 |
| 15 | 321 (FIG. 3) | 30 | 1 | King William | Neat | 3 |
| | | | | Clay | | |

The sodium chlorite solution contained in the glass ampules of the various apparatus had a sodium chlorite concentration of about 30%, with the exception of one apparatus in which a sodium chlorite concentration of about 5% was used. Several alternate reactants were also tested by filling the pouches 351 of apparatus 321 constructed in accordance with the third embodiment, as shown in FIG. 3, with a mixture containing different acid sources. In most of the apparatus, the acid source was diluted 50% in silica. However, a clay material impregnated with sulfuric acid, available from Oil-Dri of Chicago, Ill., U.S.A., under the tradename Poultry Guard, and an acid clay material commonly known as King William and available from Ralston Purina Co. of St. Louis, Mo., U.S.A., were used neat.

Each apparatus was activated and placed in a 12.8 liter glass flask. The flask was then sealed with a tight-fitting rubber stopper. A 50 ml gas tight syringe was inserted through a septum covered syringe port provided in the stopper to periodically sample the atmosphere within the flask. The sample was immediately injected into a capped, 40 ml vial containing 20 ml 1% potassium iodide (KI) and 5 ml acetic acid. The resulting iodine produced in the oxidation of the iodide by the chlorine dioxide gas was immediately titrated using sodium thiosulfate titrant and a starch indicator.

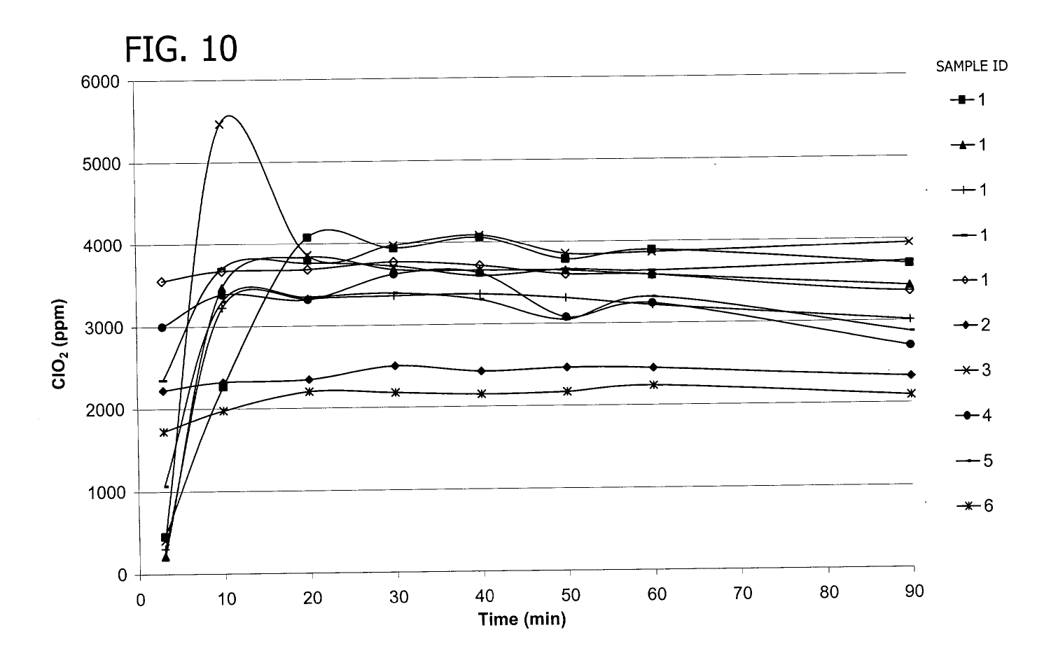

Results of the tests are shown in FIGS. 10-12. FIG. 10 is a graph of the chlorine dioxide gas concentration (ppm) over a period of ninety minutes for the different types of apparatus tested (e.g., for test samples 1-6). Several samples of the apparatus 321 shown in FIG. 3 (sample 1) were tested to evaluate the reproducibility of the chlorine dioxide gas concentration. One apparatus 721 (sample 6) constructed in accordance with the seventh embodiment as shown in FIG. 7 contained twice the reactant charge as the other apparatus types tested, but yielded a lower concentration of chlorine dioxide gas within the flask. The reduced efficiency is due to incomplete mixing in the larger apparatus. That is, with the tube of the apparatus having a larger internal cavity, such as in the range of about 6 inches×0.375 inches, the aspect ratio (e.g., about 16) was too great to allow an even distribution of the reaction components along the entire length of the tube following rupture of the ampule.

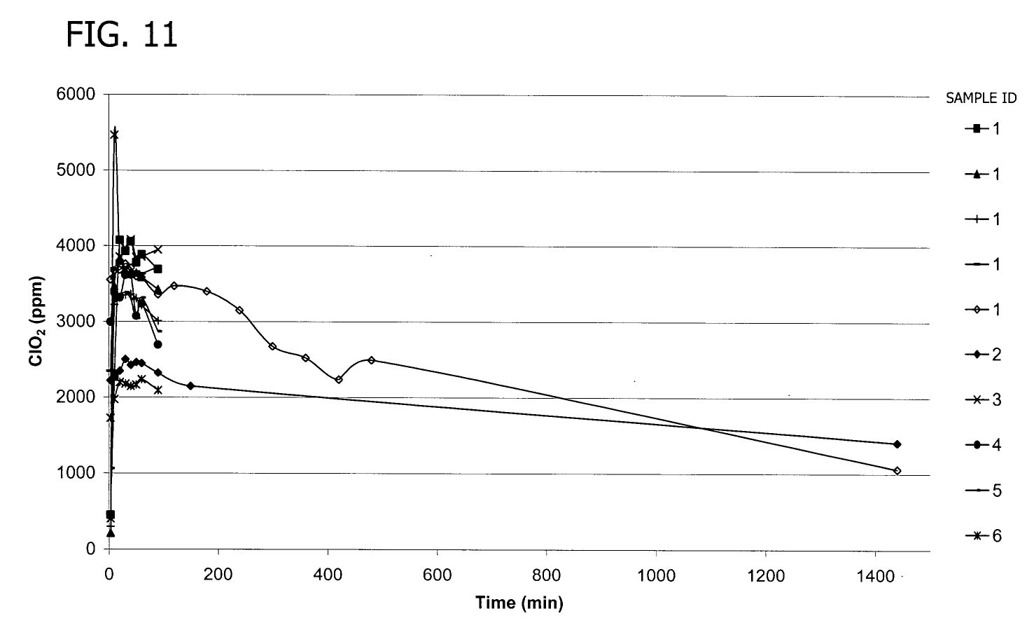

FIG. 11 is a graph of chlorine dioxide gas concentration generated by two of the tested apparatus (e.g., samples 1 and 4) over a substantially longer time period, e.g., twenty-four hours. The pouch of the apparatus tested as sample 4 was constructed of PVC to have a gas permeability substantially less than that of the Tyvek pouch of the apparatus tested as sample 1 and described previously for the apparatus 321 of FIG. 3. For the less gas permeable apparatus (sample 4), the initial concentration of chlorine dioxide gas within the flask was suppressed, with more of the chlorine dioxide gas being retained in the pouch. However, the rate at which the concentration of chlorine dioxide gas in the flask dissipated over time was lower for the less gas permeable apparatus (sample 4) due to continuous permeation of chlorine dioxide gas from the apparatus into the test volume.

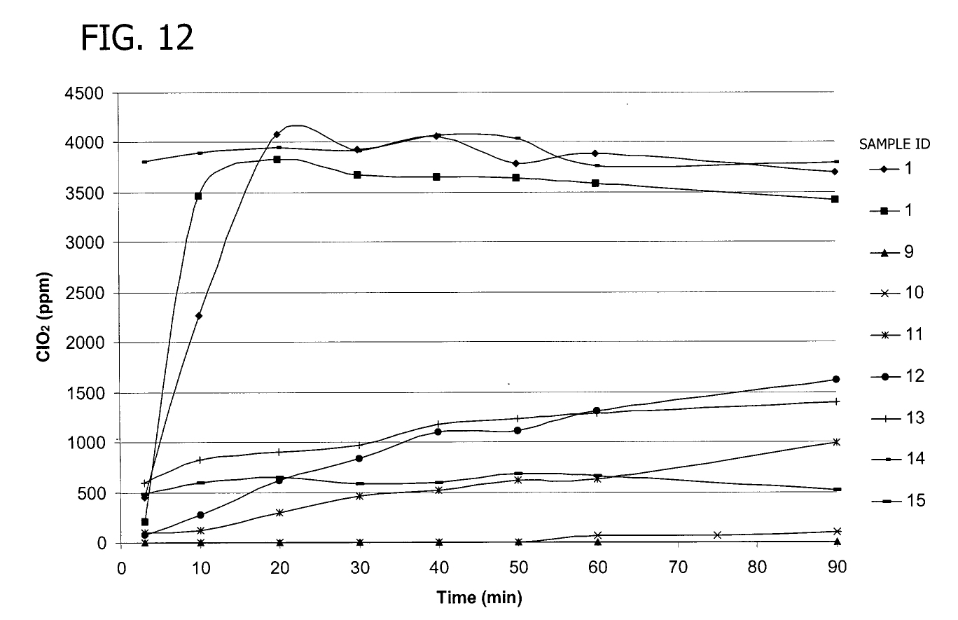

FIG. 12 is a graph of chlorine dioxide gas concentration versus time for apparatus 321 (samples 1 and 9-15) constructed in accordance with the third embodiment as shown in FIG. 3 and having different reaction components. With the exception of the Poultry Guard reaction component (sample 14), all of the tested reaction components resulted in chlorine dioxide gas generation at a rate substantially lower, and less efficiently, than the sodium persulfate mixture (sample 1). However, the Poultry Guard reaction (sample 14) was more exothermic than the sodium persulfate mixture reaction (sample 1) and may result in undesirable decomposition of the chlorine dioxide gas.

It will be recognized that the apparatus of the present invention are useful in various treatments of biologically contaminated surfaces and articles, including deodorizing, sanitizing, decontaminating and/or sterilizing such surfaces and articles. For example, in accordance with one method of the present invention for treating surfaces such as walls, furniture, machinery, etc. within an enclosure (e.g., a room), the apparatus is transported to within the enclosure in its assembled, ready-to-use form with the reaction components separately contained within the apparatus. The operator then activates the apparatus by rupturing the membrane separating the containers of the apparatus. The operator then leaves the enclosure while chlorine dioxide gas is generated by the apparatus and released into the interior of the enclosure for treating exposed surfaces therein.

In accordance with another method of the present invention, the apparatus are used to treat small articles, and in particular postal articles. In such a method, the articles to be treated are placed in a bag, and more preferably a substantially gas impermeable bag. For example, one preferred such bag is constructed of polyethylene. The operator activates the apparatus by rupturing the membrane which separates the first and second containers of the apparatus. The operator then places the activated apparatus into the bag containing the postal articles. The bag is closed, and more preferably sealed, and the chlorine dioxide gas generated and released by the apparatus fills the bag to treat the articles contained in the bag.

It is contemplated that the apparatus may instead be placed in the bag prior to being activated and then activated before or after the bag is closed without departing from the scope of this invention. For example, the bag may be constructed to have a sealable port to permit insertion of a rod therethrough for contact with the apparatus to rupture the membrane separating the containers. As another example, the membrane separating the containers of the apparatus may be ruptured by external stimuli such ultrasonic, electromagnetic or thermal stimuli.

The rate at which chlorine dioxide gas is generated and released by the apparatus into the bag containing the postal articles may be varied depending on the construction of the apparatus. Where a rapid increase in gas concentration within the bag is desired, the second container of the apparatus is preferably constructed of a generally gas permeable material. More preferably, the apparatus is constructed in accordance with the apparatus 321 of the third embodiment described above and shown in FIG. 3. Alternatively, where a slower rate of gas concentration increase is acceptable, but a decreased rate of dissipation of the gas concentration is desired, the second container of the apparatus is preferably constructed of a more gas impermeable material. For example, the apparatus may be constructed in accordance with the apparatus 221 of the second embodiment described above and shown in FIG. 2.

The apparatus of the present invention are shown and described herein as having a first container containing a first reaction component and being disposed within a second container along with a second reaction component, so that the first container broadly defines the rupturable membrane separating the reaction components. However, it is understood that other apparatus constructions may be used without departing from the scope of this invention. For example, while not shown in the drawings, the apparatus may comprise independent first and second containers respectively containing the first and second reaction components therein. Each container may be rupturable, such that the outer walls of the containers define a pair of rupturable membranes separating the reaction components. The containers may be placed in a surrounding container, such as a pouch or a tube, whereby both the first and second containers would be ruptured within the surrounding container to permit contact between the reaction components for producing chlorine dioxide gas within the surrounding container. It is also contemplated that the apparatus may comprise integrally formed first and second containers having a common outer wall that broadly defines the rupturable membrane separating the reaction components.

In view of the above, it will be seen that the several objects of the invention are achieved and other advantageous results attained. When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

As various changes could be made in the above constructions without departing from the scope of the invention, it is intended that all matter contained in the above description or shown in the accompanying drawings shall be interpreted as illustrative and not in a limiting sense.

What is claimed is:

1. Apparatus for producing chlorine dioxide gas, said apparatus comprising a first reaction component comprising a chlorite source and a second reaction component comprising an oxidizing agent, an acid releasing agent or mixtures thereof, said first and second reaction components being separated by at least one rupturable membrane whereby upon rupturing of said at least one membrane the first and second reaction components contact each other to form a reaction in which chlorine dioxide gas is produced within the apparatus, said apparatus being adapted for exhausting the chlorine dioxide gas therefrom.

2. The apparatus of claim 1 wherein the chlorite source is selected from a group consisting of: alkali metal chlorites, such as sodium chlorite or potassium chlorite; alkaline-earth metal chlorites, such as calcium chlorite; chlorite salts of a transition metal ion; a protonated primary, secondary, tertiary or quaternary amine, such as ammonium chlorite, trialkylammonium chlorite and quarternary ammonium chlorite; and, mixtures thereof.

3. The apparatus of claim 1 wherein the oxidizing agent has a stronger oxidation potential than the chlorite source.

4. The apparatus of claim 3 wherein the oxidizing agent is selected from a group consising of: persulfate; chlorine; and, mixtures thereof.

5. The apparatus of claim 1 wherein the acid releasing agent is either an acid or substance that can be hydrolyzed to form an acid.

6. The apparatus of claim 5 wherein the acid releasing agent is selected from the group consisting of: carboxylic acids; anyhydrides; acyl halides; phosphoric acid; phosphate esters; trialkylsilyl phosphate esters; dialkyl phosphates; poly phosphates; condensed phosphates; sulfonic acid; sulfonic acid esters; sulfonic acid chlorides; phosphosilicates; phosphosilicic anhydrides; carboxylates of poly α-hydroxy alcohols such as sorbitan monostearate or sorbitol monostearate; phophosiloxanes; hydrochloric acid; boric acid; citric acid; malic acid; tartaric acid; mineral acids; metal salts with acid aqueous ions such as zinc, aluminum, iron and mixtures thereof.

7. The apparatus of claim 1 wherein the first reaction component further comprises an adjuvant selected from a group consisting of: zeolite, woven, non-woven and non-powdered polymers, natural fibers, glass wool, clays, water, silica gel, metal oxides, carbides, nitrides and glass fibers and mixtures thereof.

8. The apparatus of claim 1 wherein the second reaction component further comprises an adjuvant selected from a group consisting of: zeolite, woven, non-woven and non-powdered polymers, natural fibers, glass wool, clays, water, silica gel, metal oxides, carbides, nitrides, glass fibers and mixtures thereof.

9. Apparatus as set forth in claim 1 wherein the rupturable membrane is constructed of glass.

10. Apparatus as set forth in claim 1 wherein the rupturable membrane is constructed of a polymeric material.

11. Apparatus as set forth in claim 1 wherein the membrane is rupturable upon application thereto of at least one stimuli from the group consisting of mechanical, ultrasonic, electromagnetic and thermal.

12. Apparatus as set forth in claim 1 further comprising a first container having an outer wall and containing the first reaction component therein and a second container having an outer wall and containing the second reaction component therein, at least one of the outer wall of the first container and the outer wall of the second container being rupturable to define said at least one rupturable membrane separating the first and second reaction components within said apparatus.

13. Apparatus as set forth in claim 12 wherein at least a portion of the outer wall of the first container is contained within the second container along with the second reaction component whereby said portion of the outer wall of the first container is rupturable and defines said at least one rupturable membrane separating the first and second reaction components within said apparatus.

14. Apparatus as set forth in claim 13 wherein the outer wall of the second container is substantially gas permeable to permit chlorine dioxide gas produced within said apparatus upon rupturing of said portion of the outer wall of the first container to permeate out through the outer wall of the second container for exhausting chlorine dioxide gas from said apparatus.

15. Apparatus as set forth in claim 13 wherein the second container is a pouch constructed of a substantially flexible material, the first container being contained entirely within the pouch along with the second reaction component whereby upon rupturing of the outer wall of the first container the first reaction component and the second reaction component contact each other generally within said pouch to form a reaction in which chlorine dioxide gas is produced within said pouch.

16. Apparatus as set forth in claim 15 wherein the first container is constructed of glass whereby rupturing of the first container forms glass shards within the pouch, said apparatus further comprising a protective liner intermediate the pouch and the outer wall of the first container to inhibit rupturing of the pouch by glass shards formed upon rupturing of the first container within said pouch.

17. Apparatus as set forth in claim 14 wherein the pouch is constructed of a substantially gas permeable material to permit chloride dioxide gas to diffuse therethrough for exhausting the chlorine dioxide gas from said apparatus.

18. Apparatus as set forth in claim 13 wherein the second container is generally tubular and has an internal cavity sized for receiving said portion of the outer wall of the first container along with the second reaction component.

19. Apparatus as set forth in claim 18 wherein the second container is constructed of a flexible material to permit bending thereof whereby bending of the second container applies a bending force to said portion of the outer wall of the first container to thereby rupture the first container to permit contact between the first and second reaction components within the second container.

20. Apparatus as set forth in claim 18 wherein the second container is constructed of a substantially gas permeable material to permit chlorine dioxide gas produced upon contact between the first and second reaction components to be exhausted from the apparatus by diffusing out through the outer wall of the second container.

21. Apparatus as set forth in claim 18 wherein the second container is constructed of a substantially gas impermeable material, the second container having an opening and a closure for the opening, said closure being constructed of a substantially gas permeable material to permit chlorine dioxide gas produced within said second container to be exhausted from said apparatus by diffusing out through said closure.

22. Apparatus as set forth in claim 21 wherein the closure is adapted to permit chlorine dioxide gas to be exhausted from said apparatus at a rate substantially less than a rate at which chlorine dioxide gas is generated within the apparatus.

23. A method of treating postal articles comprising the steps of:
placing at least one postal article in a bag;
activating a chlorine dioxide producing apparatus to generate chlorine dioxide gas;
placing the chlorine dioxide producing apparatus into said bag; and
closing the bag such that a concentration of chlorine dioxide gas sufficient to treat the at least one postal article fills said bag.

24. A method as set forth in claim 23 wherein the step of placing the chlorine dioxide producing apparatus into said bag is performed before the step of activating said apparatus to produce chlorine dioxide gas.

25. A method as set forth in claim 23 wherein the chlorine dioxide gas producing apparatus comprises a first reaction component, a second reaction component and at least one rupturable membrane separating the first and second reaction components, the step of activating said apparatus comprising rupturing said at least one membrane to permit contact between said first and second reaction components to facilitate a chemical reaction therebetween which produces chlorine dioxide gas within said apparatus.

26. A method as set forth in claim 25 wherein the step of rupturing the at least one membrane comprises applying at least one stimuli to said at least one membrane selected from the group comprising mechanical, ultrasonic, electromagnetic and thermal.

27. A method as set forth in claim 23 wherein the concentration of chlorine dioxide gas is sufficient to at least one of deodorize, sanitize, decontaminate, sterilize, bleach, and disinfect the at least one postal article.

28. A method of treating at least one article contained within an enclosure, said method comprising the steps of:
activating a chlorine dioxide producing apparatus to produce chlorine dioxide gas, the chlorine dioxide gas producing apparatus comprising a first reaction component contained therein and a second reaction component contained therein, said first and second reaction components being separated within said apparatus by at least one rupturable membrane, the step of activating said apparatus comprising rupturing said at least one membrane to permit contact between said first and second reaction components to facilitate a chemical reaction therebetween which produces chlorine dioxide gas within said apparatus, said apparatus being adapted for releasing chlorine dioxide gas produced therein;
placing the apparatus into the enclosure; and
closing the enclosure to permit a concentration of chloride dioxide gas produced by the apparatus sufficient to treat the at least one article to fill the enclosure.

29. A method as set forth in claim 28 wherein the step of placing the chlorine dioxide generating apparatus into said enclosure is performed before the step of activating said apparatus to generate chlorine dioxide gas.

30. A method as set forth in claim 28 wherein the enclosure is adapted for containing postal articles.

31. A method as set forth in claim 30 wherein the enclosure is a bag.

32. A method as set forth in claim 30 wherein the enclosure is a mailbox.

33. A method as set forth in claim 28 wherein the concentration of chlorine dioxide gas is sufficient to at least one of deodorize, sanitize, decontaminate, sterilize, bleach, and disinfect the at least one article.
